(19) United States
(12) Patent Application Publication
(10) Pub. No.: US 2019/0147648 A1
(43) Pub. Date: May 16, 2019

(54) Estimated 3D Models Of Interior Structures

(71) Applicant: Doug Wolff, St. Paul, MN (US)

(72) Inventor: Doug Wolff, St. Paul, MN (US)

(21) Appl. No.: 16/149,942

(22) Filed: Oct. 2, 2018

Related U.S. Application Data
(60) Provisional application No. 62/585,769, filed on Nov. 14, 2017.

(51) Int. Cl. G06T 17/20; G06T 7/70; A61C 9/00; A61B 6/14; A61B 5/055; A61B 6/03; A61C 13/00; A61C 13/34; B33Y 50/02

(52) U.S. Cl. CPC G06T 17/20; G06T 2207/30036; A61C 9/0046; A61B 6/14; A61B 5/055; A61B 6/032; A61C 13/0019; A61C 13/34; A61C 13/0004; B33Y 50/02; G06T 2210/41; G06T 2200/08; G06T 2200/04; G06T 2207/10116; G06T 2207/10088; G06T 7/70

(57) ABSTRACT

The subject matter of this specification can be embodied in, among other things, a method that includes obtaining 2D interior models of a first anatomical object, obtaining 3D surface models of the first anatomical object, obtaining 3D interior models of one or more second anatomical objects, identifying 2D locations within one or more of the 2D interior models, identifying 3D locations within one or more of the 3D surface models, identifying 3D locations within one or more of the 3D interior models, transforming one or more of the 3D interior models based on the 2D locations and the 3D locations, predicting an interior anatomical structure of the first anatomical object based on the transformed 3D interior model, and providing a predicted 3D volumetric model having a 3D surface model corresponding to the first anatomical object and a 3D anatomical interior model based on the predicted interior anatomical structure.

### CROSS-REFERENCE TO RELATED APPLICATIONS

This application claims priority to U.S. Provisional Application No. 62/585,769, filed on Nov. 14, 2017, the contents of which are incorporated herein by reference in its entirety.

### TECHNICAL FIELD

This instant specification relates to three-dimensional medical imaging.

### BACKGROUND

Medical imaging is a process in which a number of different technologies may be used in order to view structures of human or animal bodies in order to diagnose, monitor, or treat medical conditions. Dental imaging is the practice of applying medical imaging to teeth and other anatomical structures associated with the mouth.

Different types of technologies can provide different types of information about the area of the body being imaged. Photographs provide two-dimensional (2D) images of surfaces. X-rays provide 2D images that show the surface and internal structures of the subject under inspection. Magnetic resonance imaging (MRI) provides three-dimensional (3D) models of the body, but MRI machines are generally large and expensive to operate. Computerized tomography (CT) is another technique that uses multiple x-ray images to provide 3D models of the body, but CT machines are also generally large and expensive to operate and CT scanning exposes patients to large amounts of x-ray radiation. Metal in dental fillings or other prostheses can also cause artifacts in the CT image that distort the appearance of nearby internal structures of a tooth.

As technology has advanced, MRI and CT machines have become smaller and are starting to appear in dental offices for use in imaging the internal structures of patients' teeth. However, despite their usefulness and increased availability, MRI machines are still relatively expensive to use and operate, and CT scanning still exposes patients to large amounts of x-ray radiation.

### SUMMARY

In general, this document systems and techniques for performing three-dimensional medical imaging.

In a first aspect, a method for three-dimensional volumetric modeling includes obtaining one or more two-dimensional interior models of an interior of a first anatomical object of a subject, obtaining one or more three-dimensional surface models of a surface of the first anatomical object, obtaining one or more three-dimensional interior models of an interior of one or more second anatomical objects that are analogues of the first anatomical object, identifying a collection of two-dimensional locations within one or more of the two-dimensional interior models, identifying a first collection of three-dimensional locations within one or more of the three-dimensional surface models, identifying a second collection of three-dimensional locations within one or more of the three-dimensional interior models, transforming one or more of the three-dimensional interior models based on the collection of two-dimensional locations, the first collection of three-dimensional locations, and the second collection of three-dimensional locations, predicting an interior anatomical structure of the first anatomical object based on the transformed three-dimensional interior model, and providing a predicted three-dimensional volumetric model having a three-dimensional surface model corresponding to the first anatomical object and a three-dimensional anatomical interior model based on the predicted interior anatomical structure.

Various implementations can include some, all, or none of the following features. The subject can be a first subject, and the second anatomical object can be an anatomical object of a second subject that is different from the first subject. The first subject can be a dental patient, the first anatomical object can be a first tooth, the second anatomical object can be a second tooth of the second subject, and the second tooth can be an analogue of the first tooth. The first anatomical object can be the subject's analogue of the selected anatomical object. The collection of two-dimensional locations can identify locations of first anatomical landmarks on the surface and within the interior of the first anatomical object, the first collection of three-dimensional locations can identify locations of one or more of the first anatomical landmarks on the surface of the three-dimensional surface model, and the second collection of three-dimensional locations can identify locations of one or more of second anatomical landmarks on the surface and within the interior of the second anatomical object that are the second anatomical object's analogues of the first anatomical landmarks. One or more of the two-dimensional models can include a two-dimensional x-ray image. One or of the more three-dimensional surface models can include a collection of three-dimensional surface scan information provided by a three-dimensional surface scanning device. One or more of the three-dimensional interior models can include a collection of three-dimensional volumetric imaging information provided by a three-dimensional volumetric scanning device. One or more of the three-dimensional interior models can include magnetic resonance imaging data. One or more of the three-dimensional interior models can include computerized tomography scan data. The method can also include forming a tangible, three-dimensional model based on the predicted three-dimensional volumetric model. Forming a tangible, three-dimensional model can include transforming the predicted three-dimensional volumetric model into a collection of machine control parameters for a three-dimensional printer, providing the machine control parameters to the three-dimensional printer, and depositing, by the three-dimensional printer, a tangible three-dimensional arrangement of physical material based on the predicted three-dimensional volumetric model.

In a second aspect, a computer readable medium stores instructions that, when executed by one or more processors, cause the one or more processors to perform operations including obtaining one or more two-dimensional interior models of an interior of a first anatomical object of a subject, obtaining one or more three-dimensional surface models of a surface of the first anatomical object, obtaining one or more three-dimensional interior models of an interior of one or more second anatomical objects that are analogues of the first anatomical object, identifying a collection of two-dimensional locations within one or more of the two-dimensional interior models, identifying a first collection of three-dimensional locations within one or more of the three-dimensional surface models, identifying a second collection of three-dimensional locations within one or more of the three-dimensional interior models, transforming one or more of the three-dimensional interior models based on the collection of two-dimensional locations, the first collection of three-dimensional locations, and the second collection of three-dimensional locations, predicting an interior anatomical structure of the first anatomical object based on the transformed three-dimensional interior model, and providing a predicted three-dimensional volumetric model having a three-dimensional surface model corresponding to the first anatomical object and a three-dimensional anatomical interior model based on the predicted interior anatomical structure.

Various embodiments can include some, all, or none of the following features. The subject can be a first subject, and the second anatomical object can be an anatomical object of a second subject that is different from the first subject. The first subject can be a dental patient, the first anatomical object can be a first tooth, the second anatomical object can be a second tooth of the second subject, and the second tooth can be an analogue of the first tooth. The first anatomical object can be the subject's analogue of the selected anatomical object. The collection of two-dimensional locations can identify locations of first anatomical landmarks on the surface and within the interior of the first anatomical object, the first collection of three-dimensional locations can identify locations of one or more of the first anatomical landmarks on the surface of the three-dimensional surface model, and the second collection of three-dimensional locations can identify locations of one or more of second anatomical landmarks on the surface and within the interior of the second anatomical object that are the second anatomical object's analogues of the first anatomical landmarks. One or more of the two-dimensional models can include a two-dimensional x-ray image. One or of the more three-dimensional surface models can include a collection of three-dimensional surface scan information provided by a three-dimensional surface scanning device. One or more of the three-dimensional interior models can include a collection of three-dimensional volumetric imaging information provided by a three-dimensional volumetric scanning device. One or more of the three-dimensional interior models can include magnetic resonance imaging data. One or more of the three-dimensional interior models can include computerized tomography scan data. The computer readable medium can also include forming a tangible, three-dimensional model based on the predicted three-dimensional volumetric model. Forming a tangible, three-dimensional model can include transforming the predicted three-dimensional volumetric model into a collection of machine control parameters for a three-dimensional printer, providing the machine control parameters to the three-dimensional printer, and depositing, by the three-dimensional printer, a tangible three-dimensional arrangement of physical material based on the predicted three-dimensional volumetric model.

In a third aspect, a three-dimensional imaging system includes an input, an output, memory storing instructions that are executable, and one or more processing devices to execute the instructions to perform operations including obtaining one or more two-dimensional interior models of an interior of a first anatomical object of a subject, obtaining one or more three-dimensional surface models of a surface of the first anatomical object, obtaining one or more three-dimensional interior models of an interior of one or more second anatomical objects that are analogues of the first anatomical object, identifying a collection of two-dimensional locations within one or more of the two-dimensional interior models, identifying a first collection of three-dimensional locations within one or more of the three-dimensional surface models, identifying a second collection of three-dimensional locations within one or more of the three-dimensional interior models, transforming one or more of the three-dimensional interior models based on the collection of two-dimensional locations, the first collection of three-dimensional locations, and the second collection of three-dimensional locations, predicting an interior anatomical structure of the first anatomical object based on the transformed three-dimensional interior model, and providing a predicted three-dimensional volumetric model having a three-dimensional surface model corresponding to the first anatomical object and a three-dimensional anatomical interior model based on the predicted interior anatomical structure.

Various embodiments can include some, all, or none of the following features. The three-dimensional imaging system can include a storage device storing a database of three-dimensional interior models of the interiors of the second anatomical objects. The three-dimensional imaging system can include a display device, wherein providing the predicted three-dimensional volumetric model can include displaying the predicted three-dimensional volumetric model on the display device. The three-dimensional imaging system can include a communications interface, wherein obtaining one or more three-dimensional interior models can include communicating, by the communications device, a query for three-dimensional interior models to a remote database server system over a network, and receiving, by the communications device and in response to the query, one or more three-dimensional interior models of the interior of one or more second anatomical objects. The three-dimensional imaging system can include an x-ray imaging device, wherein obtaining one or more two-dimensional interior models of an interior of a first anatomical object of a subject can include imaging the first anatomical object by the x-ray imaging device. The three-dimensional imaging system can include a three-dimensional surface scanning device configured to identify three-dimensional surface, wherein obtaining one or more three-dimensional surface models of the surface of the first anatomical object can include scanning the first anatomical object by the three-dimensional surface scanning device. The three-dimensional imaging system can include a three-dimensional printer, and the operations can include transforming the predicted three-dimensional volumetric model into a collection of machine control parameters for the three-dimensional printer, providing the machine control parameters to the three-dimensional printer, and depositing, by the three-dimensional printer, a tangible three-dimensional arrangement of physical material based on the predicted three-dimensional volumetric model.

In a fourth aspect, a method for three-dimensional volumetric modeling includes obtaining a two-dimensional interior model of the interior of a first anatomical object of a subject, obtaining a three-dimensional surface model of the surface of the first anatomical object, obtaining a three-dimensional interior model of the interior of a second anatomical object of a second subject that is different from the first subject, wherein the second anatomical object is the second subject's analogues of the first anatomical object, providing a predicted three-dimensional volumetric model having a three-dimensional surface model corresponding to the first anatomical object and a three-dimensional anatomical interior model based on the two-dimensional interior model, the three-dimensional surface model, and the three-dimensional interior model.

In a fifth aspect, a method for three-dimensional volumetric modeling includes obtaining a three-dimensional interior model of the interior of an anatomical object, obtaining a two-dimensional interior model of the interior of an anatomical object, identifying a collection of two-dimensional locations within the two-dimensional interior model, identifying a collection of three-dimensional locations the three-dimensional interior model, transforming the three-dimensional interior model based on the collection of two-dimensional locations and the collection of three-dimensional locations, predicting an interior anatomical structure of the anatomical object based on the transformed three-dimensional interior model, and providing a predicted three-dimensional volumetric model having a three-dimensional surface model based on the predicted interior anatomical structure.

Various implementations can include some, all, or none of the following features. The two dimensional interior model can be modeled after a predetermined subject, the three-dimensional interior model can be modeled after the subject, and the three-dimensional interior model can be obtained prior to the two dimensional interior model. The subject can be aged a minimum of three years between when the three-dimensional interior model is obtained and when the two dimensional interior model is obtained. The method can also include obtaining a three-dimensional surface model of the surface of the anatomical object, and identifying a second collection of three-dimensional locations within the three-dimensional surface models, wherein transforming the three-dimensional interior model can be further based on the second collection of three-dimensional locations, and wherein predicting an interior anatomical structure of the anatomical object can be further based on the transformed three-dimensional interior model.

The systems and techniques described here may provide one or more of the following advantages. First, a system can provide models that estimate the internal structures of anatomical objects such as teeth. Second, the system can operate without exposing the subject to an additional exposure to CT radiation. Third, the system can operate without incurring the time and costs associated with performing an additional MRI or CT scan. Fourth, the system can provide three-dimensional models of the anatomical structures without requiring the care provider to also own and/or operate CT, MRI, or other such imaging equipment. Fifth, the system can provide information that doctors and dentists can use to enhance their diagnoses and treatments of their patients' needs.

The details of one or more implementations are set forth in the accompanying drawings and the description below. Other features and advantages will be apparent from the description and drawings, and from the claims.

### DESCRIPTION OF DRAWINGS

FIG. 1 is a schematic diagram that shows an example of a system for three-dimensional imaging.

FIG. 2 is a schematic diagram that shows another example of the system of FIG. 1.

FIG. 3 shows an example of a two-dimensional internal model.

FIGS. 4A-4C show examples of a three-dimensional surface model.

FIG. 5 shows an example of a three-dimensional internal model.

FIG. 6 shows an example of another three-dimensional internal model.

FIG. 7 shows an example anatomical model.

FIG. 8 is a conceptual block diagram of example process for three-dimensional imaging.

FIG. 9 is flow chart that shows an example of a process for three-dimensional imaging.

FIG. 10 is a schematic diagram of an example of a generic computer system

### DETAILED DESCRIPTION

This document describes systems and techniques for performing three-dimensional (3D) medical imaging. The use of two-dimensional (2D) x-rays is a common practice for obtaining simple images of a “slice” of an object, such as to see a broken bone through a patient's skin or to see how a crack or cavity has penetrated into the interior of a tooth. In existing practice, when a doctor or dentist needs to see a 3D volumetric visualization of the inside of a patient's body, magnetic resonance imaging

(MRI) or computerized tomography (CT) scanning is performed. Such techniques are expensive, and CT scans expose the patient to a large dose of x-ray radiation. With respect to dentistry, as a practical matter, such machines are simply not currently commonplace in dental offices. For these and other reasons, CT and MRI scans are generally avoided unless deemed necessary and worthwhile by the doctor or dentist. In general, the systems that will be described below can reduce or eliminate the number of MRI and/or CT scans for patients in a couple of similar ways that will be described in terms of a dental application even though they are not limited to such use.

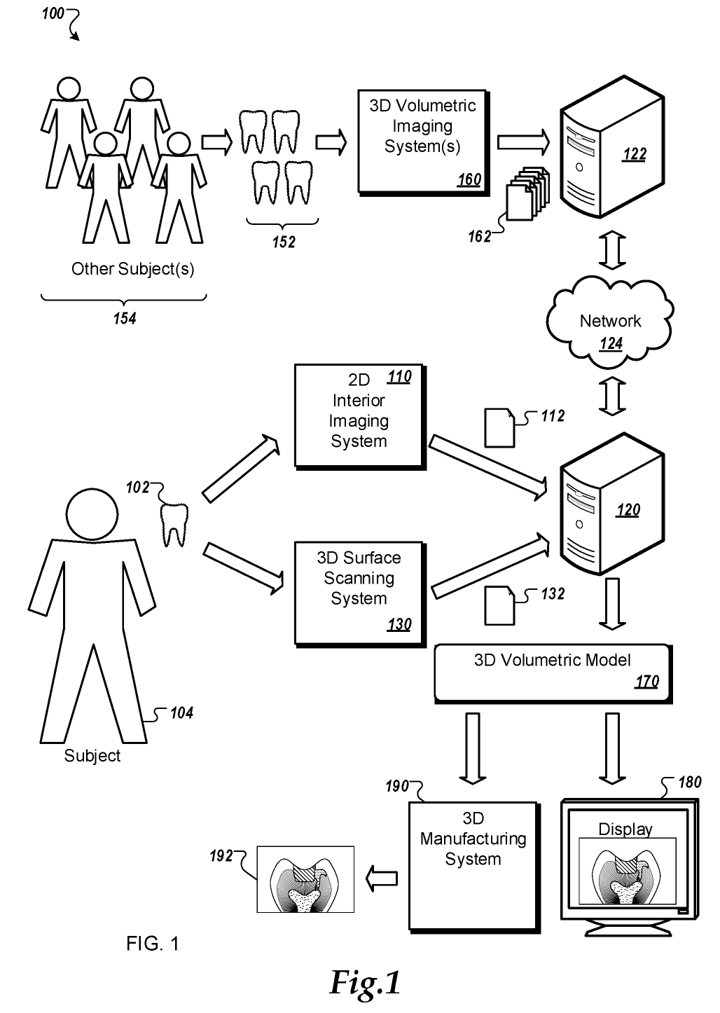

FIG. 1 is a schematic diagram that shows an example of a system 100 for three-dimensional (3D) imaging. An anatomical object 102 (e.g., a tooth) of a subject 104 (e.g., a dental patient) is identified. For example, the patent may have a lower first molar that needs care.

A 2D interior imaging system 110 (e.g., a 2D x-ray machine) is used to obtain one or more 2D interior models 112 (e.g., x-ray images) of the anatomical object and provide the models to a processing system 120 (e.g., a computer). The 2D interior models 112 describe interior and surface features (e.g., enamel, pulp, dentin) of the anatomical object 102. An operator, such as a doctor or dentist can use the processing system 120 to view the 2D interior models 112 and identify a collection of 2D locations (e.g., on the interior: enamel thickness, dentin thickness lateral borders of the pulp chamber, height of pulp horns, pulp chamber floor and pulp ceiling, on the exterior: points along the outermost edges of enamel running from the cemento-enamel junction up to occlusal table, the cusp tips, cusp slopes and occlusal fossas, other identifiable landmarks) within one or more of the 2D interior models 112. In some embodiments, the identification of locations can be done automatically (e.g., artificial intelligence) by the processing system 120.

A 3D surface scanning system 130 (e.g., a handheld 3D scanner) is used to obtain one or more 3D surface models 132 (e.g., 3D scans) of the anatomical object and provide the models to the processing system 120. The 3D surface models 132 describe surface features (e.g., the outer shape) of the anatomical object 102. The operator can use the processing system 120 to view the 3D surface models 132 and identify a collection of 3D locations of surface locations described by one or more of the 3D surface models 132. At least some of the identified locations are locations that were also identified in the 2D interior models 112. In some embodiments, the identification of locations can be done automatically (e.g., artificial intelligence) by the processing system 120.

The processing system 120 is in data communication with a server system 122 over a communication network 124 (e.g., the Internet). The server system 122 includes a database or other data store of existing 3D volumetric models 162 (e.g., MRI and/or CT scans). The existing 3D volumetric models 162 represent various scans of one or more subjects 154 other than the subject 104. The processing system 120 queries the server system 122 to obtain one or more of the existing 3D volumetric models 162 that correspond to a collection of anatomical objects 152 that correspond to the anatomical object 102. In the illustrated example, the subjects 154 have been scanned by one or more 3D volumetric imaging systems 160 (e.g., MRI, CT, micro CT), and the resulting scan data can include images of the subjects' teeth, including teeth that are anatomically equivalent to those of the subject 104. For example, the upper right 1st molar of the subject 104 may be in need of care, and the processing system 120 can query the server system 122 for existing MRI and/or CT scans upper right 1st molars of other patients.

In some embodiments, the 3D volumetric models 162 can be obtained as by-products of other procedures that have been performed on the other subjects. For example, one of the other subjects 154 may have been scanned to diagnose a broken jaw, while another of the other subjects 154 may have been scanned to diagnose a sinus problem, while yet another of the other subjects 154 may be cadavers of individuals who donated their bodies to science, while yet another of the subjects 154 may have been scanned for other dental procedures like dental implants, oral surgery or orthodontics.

In some embodiments, the 3D volumetric models 162 may be anonymized to prevent the identification of the individuals from whom the 3D volumetric models 162 were obtained. In some embodiments, the server system 122 or the processing system 120 can combine (e.g., average, normalize) multiple 3D volumetric models 162 to create a combined model that is representative of selected anatomical objects, such that the processing system 120 is provided with a 3D volumetric model that is not representative of any individual one of the other subjects 154.

The 3D volumetric models 162 describe interior and surface features (e.g., enamel, pulp, dentin) of the anatomical objects 152. The operator can use the processing system 120 to view the 3D volumetric models 162 and identify a collection of 3D locations within one or more of the 3D volumetric models 162. At least some of the identified locations are locations that were also identified in the 2D interior models 112 and the 3D surface scan models 132. In some embodiments, the identification of locations can be done automatically (e.g., artificial intelligence) by the processing system 120, the server system 122, or other systems.

The processing system 120 processes the 2D interior models 112, the 3D surface scan models 132, the 3D volumetric models 162, and the locations identified from the models 112, 132, and 162 to transform a 3D interior model that predicts a 3D volumetric model 170 of the interior anatomical structure of the anatomical object 102 based on the transformed 3D interior model. For example, the processing system can modify (e.g., morph) one or more of the 3D volumetric models 162 to cause the 3D features of the 3D volumetric model(s) 162 to have surface shapes that resemble those of the anatomical object 102 and interior feature shapes that resemble those of the anatomical object 102.

For example, large data sets of dental imagery and models can be used to predict the morphology of the internal dental pulp, such as pulp chamber height, pulpal floor, width, and pulp horns based on the scanned external morphology of the tooth, including height, width in both mesial-distal and bucco-lingual directions, cusps, fossae, secondary grooves, ridges, pits and other unique dental morphological features. Then using one or more 2D radiographs coupled with large data sets, the 2D thickness of the enamel, the thickness of the dentin layer, and the volume of the dental pulp can be estimated. The 3D and 2D imagery and models can then meshed into a 3D representation of the internal enamel thickness, dentin thickness, and/or pulp tissue morphology, including features such as the chamber height, pulpal floor, width of pulp chamber in mesial-distal and/or bucco-lingual dimensions, and the height of pulp horns.

The 3D volumetric model 170 is provided as a predicted 3D volumetric model having a 3D surface model corresponding to the anatomical object 102 and a 3D anatomical interior model based on the predicted interior anatomical structure. A display 180 is used to provide the 3D volumetric model 170 as a simulated MRI or CT scan of the anatomical object 102. A 3D manufacturing system 190 (e.g., a 3D printer or other computer-aided manufacturing system) is used to manufacture a tangible, physical 3D model 192 of the 3D volumetric model 170.

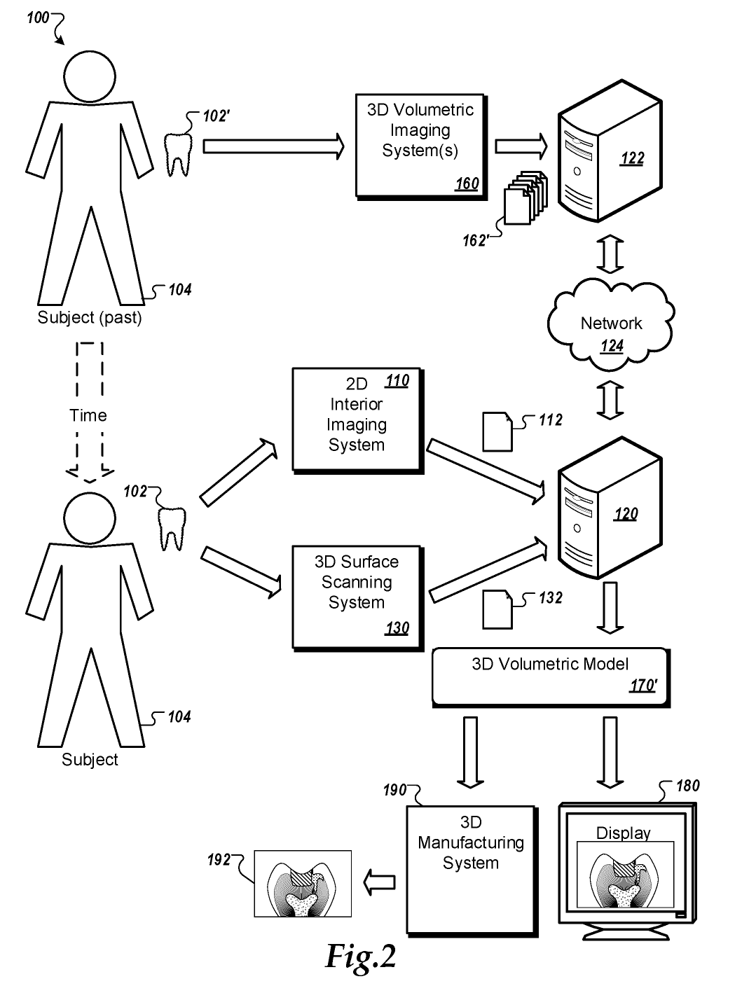

FIG. 2 is a schematic diagram that shows another example of the system 100 of FIG. 1. As in the example of FIG. 1, the anatomical object 102 (e.g., a tooth) of the subject 104 (e.g., a dental patient) is identified. However, in the example of FIG. 2, instead of using 3D volumetric models 162 obtained from the other subjects 134, one or more older 3D volumetric models 162′ of the subject's 104 anatomical object 102 as it appeared in the past, designated as 102′ is used by the processing system 120 to determine a 3D volumetric model 170′.

As some anatomical objects age, their interior and exterior shapes can change and grow. For example, as the dental pulp ages, it recedes in volume due to factors such as normal physiology and in response to external stimuli such as decay, dental restorations, orthodontic treatment, trauma, and attrition that stimulate reparative dentin production. The increased thickness of the dentin layer results in a corresponding reduction in the pulpal volume.

As such, MRI, CT, or other such 3D volumetric scans taken of the anatomical object 102′ sufficiently long ago may not be completely representative of the current interior structure of the anatomical object 102. The processing system 120 is used to transform the subject's 104 own older 3D volumetric model(s) 162′ to create a 3D volumetric model 170′ that estimates the current internal structure of the anatomical object 102, based on the current surface and interior locations of the 2D interior model 112, the current 3D surface model 132, and the surface and interior locations of the 3D volumetric models 162′. The 3D volumetric model 170′ is presented on the display 180 and/or manufactured using the 3D manufacturing system 190.

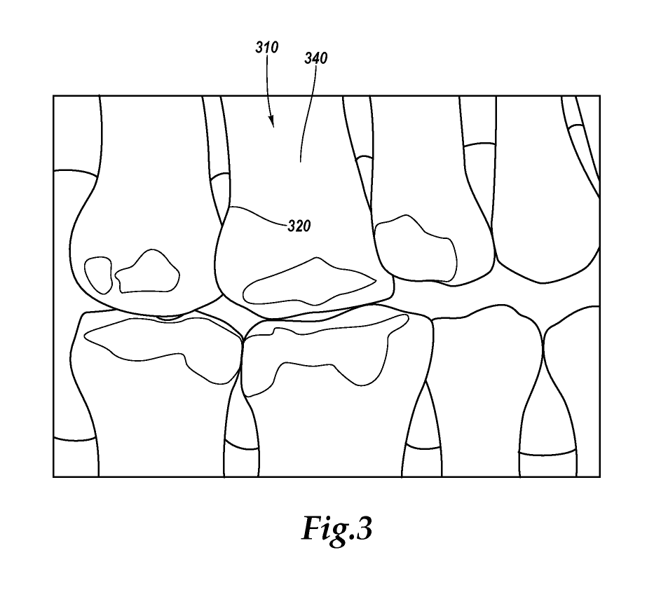

FIG. 3 shows an example of a 2D internal model 300. In some embodiments, the 2D internal model 300 can be the example 2D internal model 112 of FIGS. 1 and 2. In the illustrated example, the 2D internal model 300 is a two-dimensional dental X-ray. The 2D internal model 300 includes information that describes a tooth 310, including a surface anatomy 320 (e.g., external points along the mesial and distal surfaces, cups heights, cusp slopes and fossae) and an interior anatomy 340 (e.g., enamel thickness, dentin thickness, lateral borders of the pulp chamber, height of pulp horns, pulp chamber floor, pulp ceiling).

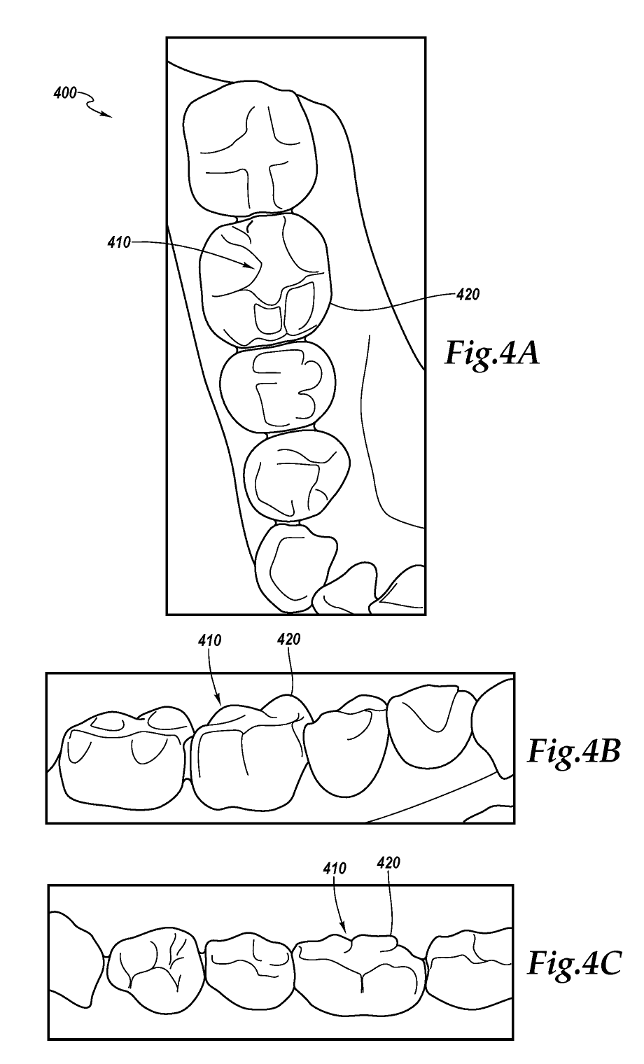

In some embodiments, the tooth 410 can be the example anatomical object 102. FIGS. 4A-4C show examples of a 3D surface model 400. In some embodiments, the 3D surface model 400 can be the example 3D surface model 132 of FIGS. 1 and 2. In the illustrated example, the 3D surface model 400 is a three-dimensional dental surface scan. The 3D surface model 400 includes information that describes a tooth 410, including a surface anatomy 420 (e.g., buccal, lingual, mesial and distal contour, width in both mesial-distal and bucco-lingual directions, cusps, fossae, secondary grooves, pits, ridges, other unique dental anatomical features). In some embodiments, the tooth 410 can be the example tooth 310 of FIG. 3 and/or can be the example anatomical object 102 of FIGS. 1 and 2.

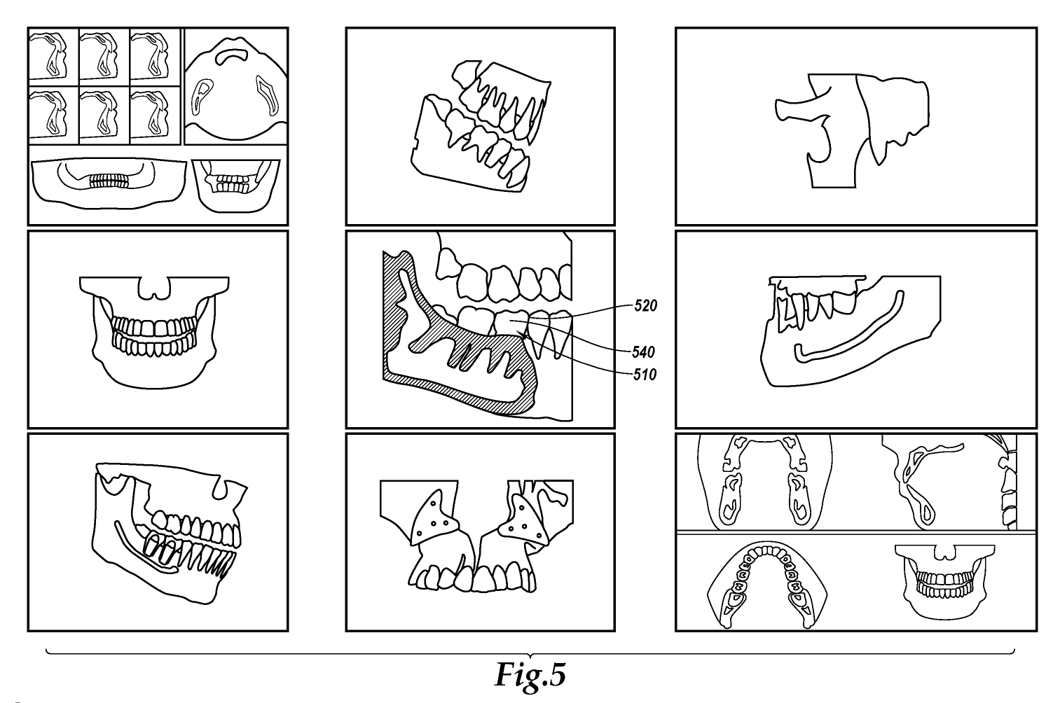

FIG. 5 shows an example of a 3D internal model 500. In some embodiments, the 3D internal model 500 can be one or more of the example 3D internal models 162 of FIGS. 1 and 2. In the illustrated example, the 3D internal model 500 is a three-dimensional volumetric CT scan. The 3D internal model 500 includes information that describes a tooth 510, including a surface anatomy 520 (e.g., buccal, lingual, mesial and distal contour, width in both mesial-distal and bucco-lingual directions, cusps, fossae, secondary grooves, pits, ridges, other unique dental anatomical features), and an interior anatomy 540 (e.g., enamel thickness, dentin thickness lateral borders of the pulp chamber, height of pulp horns, pulp chamber floor, pulp ceiling). In some embodiments, the tooth 510 can be the example anatomical object 102′, or more of the example anatomical objects 152.

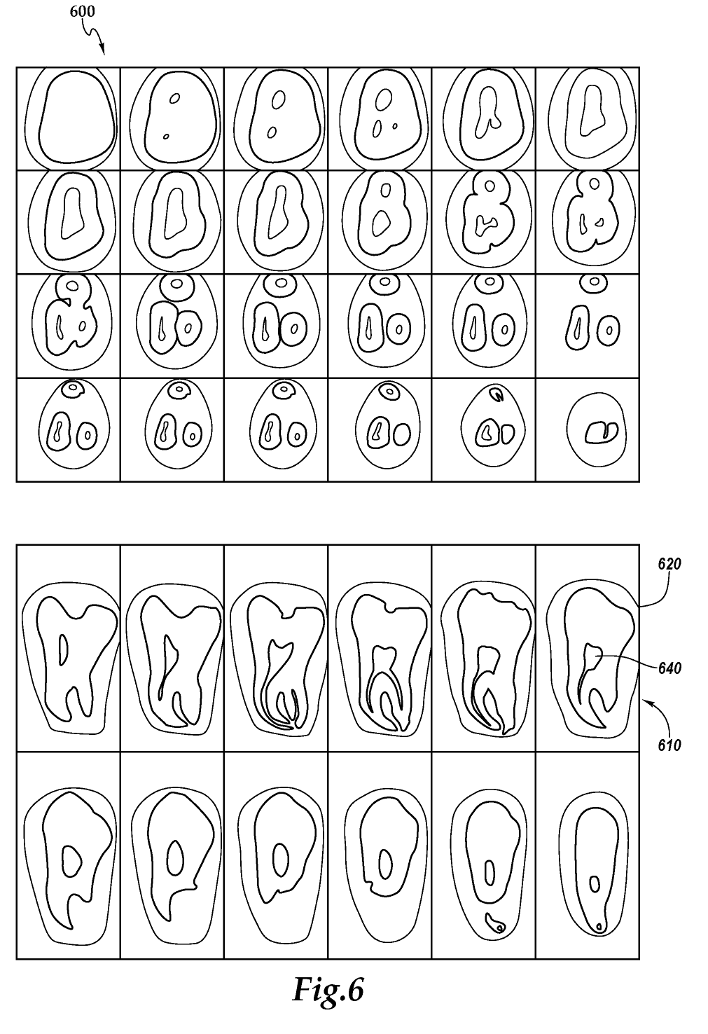

FIG. 6 shows an example of a 3D internal model 600. In some embodiments, the 3D internal model 600 can be one or more of the example 3D internal models 162 of FIGS. 1 and 2. In the illustrated example, the 3D internal model 600 is a three-dimensional volumetric MRI scan. The 3D internal model 600 includes information that describes a tooth 610, including a surface anatomy 620 (e.g., buccal, lingual, mesial and distal contour, width in both mesial-distal and bucco-lingual directions, cusps, fossae, secondary grooves, pits, ridges, other unique dental anatomical features) and an interior anatomy 640 (e.g., enamel thickness, dentin thickness lateral borders of the pulp chamber, height of pulp horns, pulp chamber floor and pulp ceiling). In some embodiments, the tooth 610 can be the example anatomical object 102′, or more of the example anatomical objects 152.

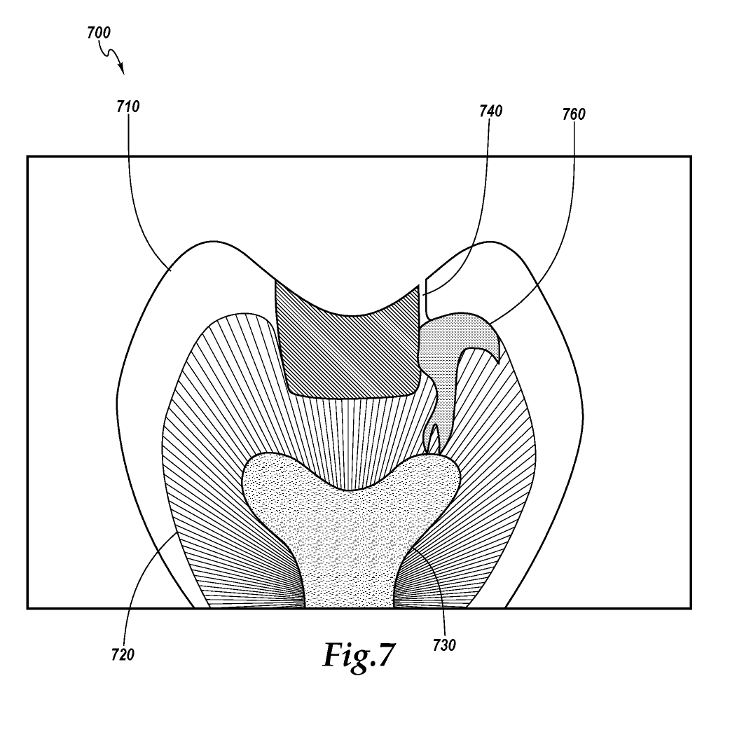

FIG. 7 shows an example anatomical model 700. In some embodiments, the anatomical model 700 can be the example 3D volumetric model 170 of FIG. 1 or 170′ of FIG. 2. The anatomical model 700 includes surface and internal anatomical features such as an enamel layer 710, a dentin layer 720, and a pulp layer 730. The anatomical model 700 also includes features such as a leaking gap 740 around a filling 750 and secondary caries 760. In some embodiments, the anatomical model 700 can be estimated based on one or a combination of 3D internal models. For example, a collection of CT scans, MRI scans, and/or similar 3D volumetric information for a selected tooth type can be averaged, normalized, or otherwise combined to create a 3D volumetric model that generally represents the surface and internal geometries of the selected type of tooth. In some implementations, the model may be derived from 3D models taken from subjects of approximately the same age. For example, as a person ages, pulp chamber volume can decrease due to normal physiological changes. In addition, the deposition of reparative dentin because of things such as caries, dental restorative procedures, orthodontic treatments, trauma, and attrition can contribute to a decrease in pulp chamber size and/or a corresponding increase in the thickness of the dentin layer. As such, a 3D volumetric model may be derived from a collection of 3D internal scans taken of subjects of a similar age, to provide a normalized representation of a typical tooth or other structure of that age.

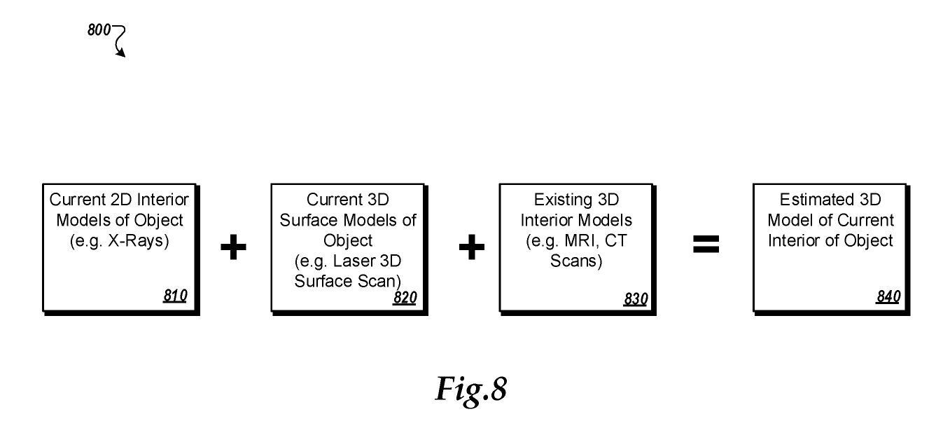

FIG. 8 is a conceptual block diagram of example process 800 for three-dimensional imaging. In some embodiments, the process 800 can be performed by the all or part of the system 100 of FIGS. 1 and 2.

At 810, one or more current (e.g., recent, contemporary) 2D interior models of an anatomical object are obtained. For example, traditional 2D dental x-rays of a selected tooth of a particular patent may be obtained. In some implementations, the 2D interior models can be the 2D interior models 112.

At 820, one or more current (e.g., recent, contemporary) 3D surface models of the same anatomical object are obtained. For example, a handheld 3D surface scanner can be used to measure the contours and dimensions of the surfaces of the selected tooth of the particular patent. In some implementations, the 3D surface models can be the 3D surface models 132.

At 830, one or more existing 3D interior models are obtained. In some embodiments, the existing 3D interior models can be the 3D interior models 162. In some embodiments, the existing 3D interior models can be new or older MRI, CT, or other 3D volumetric scans of patients other than the subject being diagnosed. In some embodiments, the existing 3D interior models can be older MRI, CT, or other 3D volumetric scans of the subject being diagnosed (e.g., an MRI scan of a patent that was taken several months or years in the past, possibly as part of a diagnosis for a different issue).

The three types of scans are combined by identifying 2D and 3D locations of anatomical features that are common among two or more of the scan types. For example, the locations of points along the external layer of enamel starting from the cemento-enamel junction up to the occlusal table, the cusp heights, the cusp slopes, and the occlusal fossae can be identified in 2D x-rays of a patient's tooth, and those same points along the external layer of enamel starting from the cemento-enamel junction up to the occlusal table, the cusp heights, the cusp slopes, and the occlusal fossae can be identified on 3D surface scans of the patient's tooth. Equivalent points in the detailed occlusal anatomy, cusps, ridges, grooves, fossae, pits, contours of the buccal, lingual, mesial and distal surfaces can also be identified on a 3D volumetric model of a tooth. Locations of internal features such as enamel thickness, dentin thickness, pulp chamber height, pulp chamber floor, lateral borders of the pulp chamber, and the height of the pulp horns can also be identified in the 2D x-rays of the patient's tooth, and equivalent internal features can also be identified in the 3D volumetric model.

The processing system 120 can modify the 3D volumetric model to cause the internal and external locations of features identified within the 3D volumetric model to more closely emulate their actual or estimated locations in the actual tooth. As such, 2D internal images and 3D surface images can be used to predict the internal structures of the patient's tooth such as enamel thickness, dentin thickness, pulp chamber height, pulp chamber floor, width of pulp chamber in mesial-distal and/or bucco-lingual dimensions, and the height of the pulp horns without requiring the patent to undergo a first or additional MRI, CT, or other such 3D volumetric imaging process.

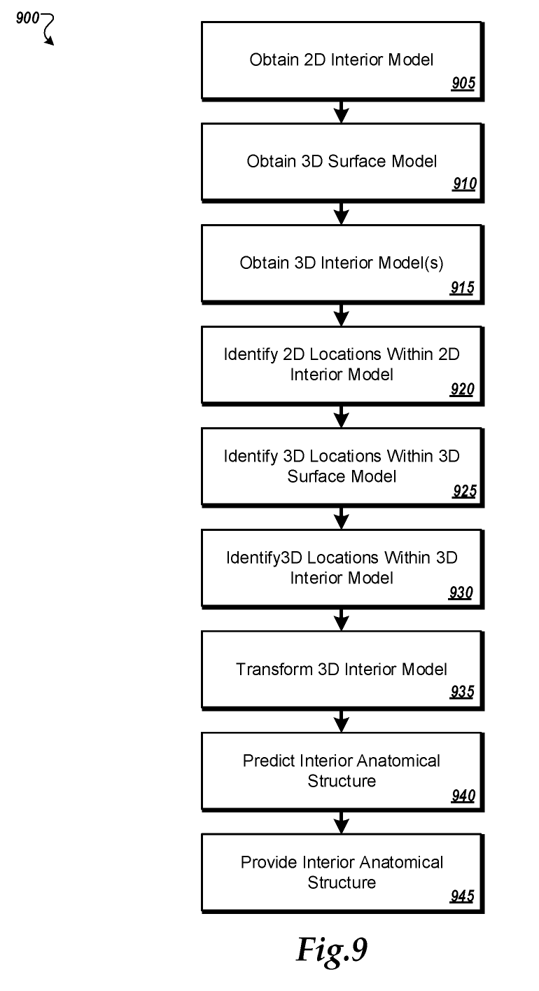

FIG. 9 is flow chart that shows an example of a process 900 for three-dimensional imaging. In some implementations, the process 900 can be performed by part (e.g., the processing system 120) or all of the system 100 of FIGS. 1 and 2.

At 905, one or more 2D interior models of an interior of a first anatomical object of a subject are obtained. In some implementations, one or more of the 2D models can be a 2D x-ray image. For example, one or more of the example 2D interior models 112 can be obtained (e.g., 2D x-rays of the anatomical object 102 (e.g., tooth) of the subject 104 can be taken).

At 910, one or more 3D surface models of a surface of the first anatomical object are obtained. In some implementations, one or of the more 3D surface models can be a collection of 3D surface scan information provided by a 3D surface scanning device. For example, one or more of the example 3D surface models 132 can be obtained by inserting a handheld 3D scanner into the subject's 104 mouth to map the surface geometry of the subject's 104 anatomical object 102 (e.g., tooth).

At 915, one or more 3D interior models of an interior of one or more second anatomical objects that are analogues of the first anatomical object are obtained. For example, one or more of the example 3D volumetric models 162 and/or 162′ can be downloaded from the server system 122.

In some implementations, one or more of the 3D interior models can be a collection of 3D volumetric imaging information provided by a 3D volumetric scanning device. In some implementations, one or more of the 3D interior models can be magnetic resonance imaging (MRI) data. In some implementations, one or more of the 3D interior models can be computerized tomography (CT) scan data.

In some implementations, the subject can be a first subject, and the second anatomical object can be an anatomical object of a second subject that is different from the first subject. In some implementations, the first subject can be a dental patient, the first anatomical object can be a first tooth, the second anatomical object can be a second tooth of the second subject, and the second tooth can be an analogue of the first tooth. For example, the example subject 104 can be dental patient “Doug”, the anatomical object 102 can be Doug's upper left lateral incisor, and the second anatomical objects 152 can be upper left lateral incisors of example subjects 154 “Bob”, “Stuart”, and “Trevor”.

In some implementations, the first anatomical object can be the subject's analogue of the selected anatomical object. For example, the example anatomical object 102 can be Doug's upper left lateral incisor as it exists today, while the example anatomical object 102′ can be Doug's upper left lateral incisor as it existed as some point weeks, months, or years in the past.

At 920, a collection of 2D locations within one or more of the 2D interior models is identified. For example, features such as external points along the mesial and distal surfaces, cups heights, cusp slopes, fossae, and/or other unique dental anatomical features can identified by a doctor, dentist, or artificial intelligence system.

At 925, a first collection of 3D locations within one or more of the 3D surface models is identified. For example, features such as buccal, lingual, mesial and distal contour, width in both mesial-distal and bucco-lingual directions, cusps, fossae, secondary grooves, pits, ridges, and/or other unique dental anatomical features can identified by a doctor, dentist, or artificial intelligence system.

At 930, a second collection of 3D locations within one or more of the 3D interior models is identified. For example, features such as external points along the mesial and distal surfaces, cups heights, cusp slopes, fossae, buccal, lingual, mesial and distal contour, width in both mesial-distal and bucco-lingual directions, cusps, fossae, secondary grooves, pits, ridges, and/or other unique dental anatomical features can identified by a doctor, dentist, or artificial intelligence system.

In some implementations, the collection of 2D locations can identify locations of first anatomical landmarks on the surface and within the interior of the first anatomical object, the first collection of 3D locations can identify locations of one or more of the first anatomical landmarks on the surface of the 3D surface model, and the second collection of 3D locations can identify locations of one or more of second anatomical landmarks on the surface and within the interior of the second anatomical object that are the second anatomical object's analogues of the first anatomical landmarks. For example, a dentist or the processing system 120 can identify the 2D and 3D locations of selected anatomical features that are visible in two or more of the 2D interior models 112, the 3D surface models 132, and the 3D interior models 162.

At 935, one or more of the 3D interior models is transformed based on the collection of 2D locations, the first collection of 3D locations, and the second collection of 3D locations. For example, the internal and external geometries of one or more of the example 3D interior models 162 and/or 162′ can be modified to make the 3D interior models 162 and/or 162′ more closely align with corresponding internal and external features of the example anatomical object 102 as identified in the 2D internal models 112 and the 3D surface models 132.

At 940, an interior anatomical structure of the first anatomical object is predicted based on the transformed 3D interior model. For example, the transformed 3D interior model can describe an interior structure that predicts, emulates, or otherwise estimates the internal anatomical structure of the anatomical object 102.

At 945, a predicted 3D volumetric model having a 3D surface model corresponding to the first anatomical object and a 3D anatomical interior model is predicted based on the predicted interior anatomical structure. For example, the example 3D volumetric models 170 and/or 170′ can be provided, in which the 3D volumetric models 170, 170′ describe external surface features that are representative of the external surface features of the anatomical object 102 and describe internal anatomical features that are predictive of the internal anatomical features of the anatomical object 102.

In some implementations, the process 900 can also include forming a tangible, 3D model based on the predicted 3D volumetric model. In some implementations, forming a tangible, 3D model can include transforming the predicted 3D volumetric model into a collection of machine control parameters for a 3D printer, providing the machine control parameters to the 3D printer, and depositing, by the 3D printer, a tangible 3D arrangement of physical materials based on the predicted 3D volumetric model. For example, the 3D volumetric model 170 and/or 170′ can be turned into a physical model by the 3D manufacturing system 190. In some examples, the resulting physical model can made of biocompatible materials suitable for use as a prosthetic (e.g., a dental cap, crown, bridge, or implant). In some examples, the resulting physical model can be an enlargement of the anatomical object (e.g., a “giant” polystyrene foam tooth having differently colored internal layers that correspond to different anatomical features that a dentist can cut open with a crafter's knife to explain or rehearse an upcoming dental procedure for a patient).

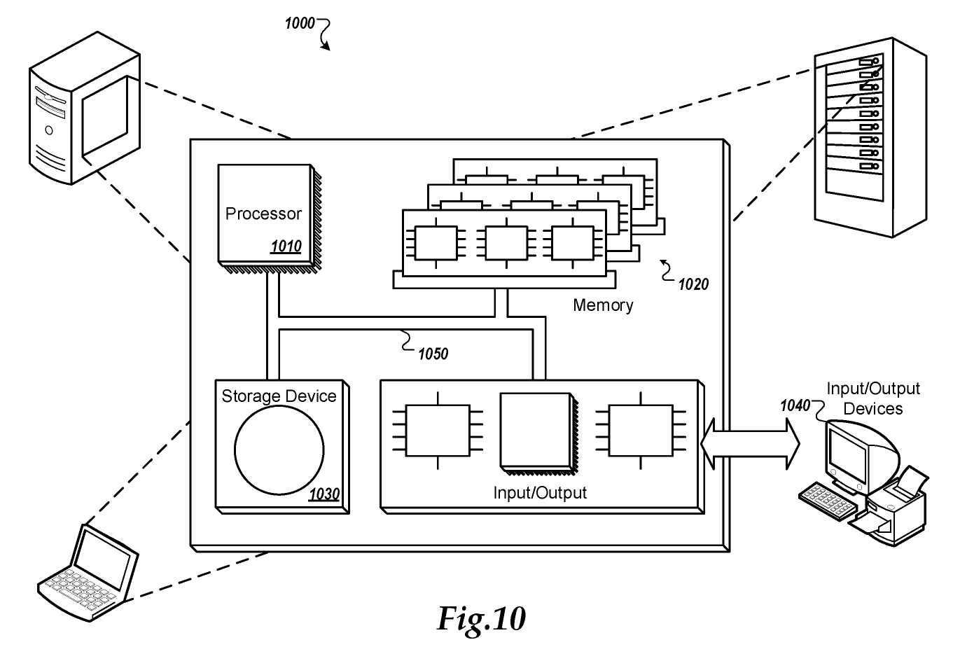

FIG. 10 is a schematic diagram of an example of a generic computer system 1000. The system 1000 can be used for the operations described in association with the method 300 according to one implementation. For example, the system 1000 may be included in either or all of the processing system 120 and/or the server system 122 of FIGS. 1 and 2.

The system 1000 includes a processor 1010, a memory 1020, a storage device 1030, and an input/output device 1040. Each of the components 1010, 1020, 1030, and 1040 are interconnected using a system bus 1050. The processor 1010 is capable of processing instructions for execution within the system 1000. In one implementation, the processor 1010 is a single-threaded processor. In another implementation, the processor 1010 is a multi-threaded processor. The processor 1010 is capable of processing instructions stored in the memory 1020 or on the storage device 1030 to display graphical information for a user interface on the input/output device 1040.

The memory 1020 stores information within the system 1000. In one implementation, the memory 1020 is a computer-readable medium. In one implementation, the memory 1020 is a volatile memory unit. In another implementation, the memory 1020 is a non-volatile memory unit.

The storage device 1030 is capable of providing mass storage for the system 1000. In one implementation, the storage device 1030 is a computer-readable medium. In various different implementations, the storage device 1030 may be a floppy disk device, a hard disk device, an optical disk device, or a tape device.

The input/output device 1040 provides input/output operations for the system 1000. In one implementation, the input/output device 1040 includes a keyboard and/or pointing device. In another implementation, the input/output device 1040 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

Although a few implementations have been described in detail above, other modifications are possible. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

What is claimed is:

1. A method for three-dimensional volumetric modeling, comprising:
obtaining one or more two-dimensional interior models of an interior of a first anatomical object of a subject;
obtaining one or more three-dimensional surface models of a surface of the first anatomical object;
obtaining one or more three-dimensional interior models of an interior of one or more second anatomical objects that are analogues of the first anatomical object;
identifying a collection of two-dimensional locations within one or more of the two-dimensional interior models;
identifying a first collection of three-dimensional locations within one or more of the three-dimensional surface models;
identifying a second collection of three-dimensional locations within one or more of the three-dimensional interior models;
transforming one or more of the three-dimensional interior models based on the collection of two-dimensional locations, the first collection of three-dimensional locations, and the second collection of three-dimensional locations;
predicting an interior anatomical structure of the first anatomical object based on the transformed three-dimensional interior model; and
providing a predicted three-dimensional volumetric model having a three-dimensional surface model corresponding to the first anatomical object and a three-dimensional anatomical interior model based on the predicted interior anatomical structure.

2. The method of claim 1, wherein the subject is a first subject, and the second anatomical object is an anatomical object of a second subject that is different from the first subject.

3. The method of claim 2, wherein the first subject is a dental patient, the first anatomical object is a first tooth, the second anatomical object is a second tooth of the second subject, and the second tooth is an analogue of the first tooth.

4. The method of claim 1, wherein:
the collection of two-dimensional locations identify locations of first anatomical landmarks on the surface and within the interior of the first anatomical object;
the first collection of three-dimensional locations identify locations of one or more of the first anatomical landmarks on the surface of the three-dimensional surface model; and
the second collection of three-dimensional locations identify locations of one or more of second anatomical landmarks on the surface and within the interior of the second anatomical object that are the second anatomical object's analogues of the first anatomical landmarks.

5. The method of claim 1, wherein one or more of the two-dimensional models comprises a two-dimensional x-ray image.

6. The method of claim 1, wherein one or of the more three-dimensional surface models comprises a collection of three-dimensional surface scan information provided by a three-dimensional surface scanning device.

7. The method of claim 1, wherein one or more of the three-dimensional interior models comprises a collection of three-dimensional volumetric imaging information provided by a three-dimensional volumetric scanning device, or magnetic resonance imaging data, or computerized tomography scan data.

8. The method of claim 1, further comprising:
transforming the predicted three-dimensional volumetric model into a collection of machine control parameters for a three-dimensional printer;
providing the machine control parameters to the three-dimensional printer; and
depositing, by the three-dimensional printer, a tangible three-dimensional arrangement of physical material based on the predicted three-dimensional volumetric model.

9. A computer readable medium storing instructions that, when executed by one or more processors, cause the one or more processors to perform operations comprising:
obtaining one or more two-dimensional interior models of an interior of a first anatomical object of a subject;
obtaining one or more three-dimensional surface models of a surface of the first anatomical object;
obtaining one or more three-dimensional interior models of an interior of one or more second anatomical objects that are analogues of the first anatomical object;
identifying a collection of two-dimensional locations within one or more of the two-dimensional interior models;
identifying a first collection of three-dimensional locations within one or more of the three-dimensional surface models;
identifying a second collection of three-dimensional locations within one or more of the three-dimensional interior models;
transforming one or more of the three-dimensional interior models based on the collection of two-dimensional locations, the first collection of three-dimensional locations, and the second collection of three-dimensional locations;
predicting an interior anatomical structure of the first anatomical object based on the transformed three-dimensional interior model; and
providing a predicted three-dimensional volumetric model having a three-dimensional surface model corresponding to the first anatomical object and a three-dimensional anatomical interior model based on the predicted interior anatomical structure.

10. The computer readable medium of claim 9, wherein the subject is a first subject and the second anatomical object is an anatomical object of a second subject that is different from the first subject.

11. The computer readable medium of claim 10, wherein the first subject is a dental patient, the first anatomical object is a first tooth, the second anatomical object is a second tooth of the second subject, and the second tooth is an analogue of the first tooth.

12. The computer readable medium of claim 9, wherein:
the collection of two-dimensional locations identify locations of first anatomical landmarks on the surface and within the interior of the first anatomical object;
the first collection of three-dimensional locations identify locations of one or more of the first anatomical landmarks on the surface of the three-dimensional surface model; and
the second collection of three-dimensional locations identify locations of one or more of second anatomical landmarks on the surface and within the interior of the second anatomical object that are the second anatomical object's analogues of the first anatomical landmarks.

13. The computer readable medium of claim 9, wherein one or more of the two-dimensional models comprises a two-dimensional x-ray image.

14. The computer readable medium of claim 9, wherein one or of the more three-dimensional surface models comprises a collection of three-dimensional surface scan information provided by a three-dimensional surface scanning device.

15. The computer readable medium of claim 9, wherein one or more of the three-dimensional interior models comprises a collection of three-dimensional volumetric imaging information provided by a three-dimensional volumetric scanning device, or magnetic resonance imaging data, or computerized tomography scan data.

16. The computer readable medium of claim 9, further comprising:
transforming the predicted three-dimensional volumetric model into a collection of machine control parameters for a three-dimensional printer;
providing the machine control parameters to the three-dimensional printer; and
depositing, by the three-dimensional printer, a tangible three-dimensional arrangement of physical material based on the predicted three-dimensional volumetric model.

17. A three-dimensional imaging system, comprising:
an input;
an output;
memory storing instructions that are executable; and
one or more processing devices to execute the instructions to perform operations comprising:
obtaining one or more two-dimensional interior models of an interior of a first anatomical object of a subject;
obtaining one or more three-dimensional surface models of a surface of the first anatomical object;
obtaining one or more three-dimensional interior models of an interior of one or more second anatomical objects that are analogues of the first anatomical object;
identifying a collection of two-dimensional locations within one or more of the two-dimensional interior models;
identifying a first collection of three-dimensional locations within one or more of the three-dimensional surface models;
identifying a second collection of three-dimensional locations within one or more of the three-dimensional interior models;
transforming one or more of the three-dimensional interior models based on the collection of two-dimensional locations, the first collection of three-dimensional locations, and the second collection of three-dimensional locations;
predicting an interior anatomical structure of the first anatomical object based on the transformed three-dimensional interior model; and
providing a predicted three-dimensional volumetric model having a three-dimensional surface model corresponding to the first anatomical object and a three-dimensional anatomical interior model based on the predicted interior anatomical structure.

18. The three-dimensional imaging system of claim 17, further comprising an x-ray imaging device, wherein obtaining one or more two-dimensional interior models of an interior of a first anatomical object of a subject further comprises imaging the first anatomical object by the x-ray imaging device.

19. The three-dimensional imaging system of claim 17, further comprising a three-dimensional surface scanning device configured to identify three-dimensional surface, wherein obtaining one or more three-dimensional surface models of the surface of the first anatomical object comprises scanning the first anatomical object by the three-dimensional surface scanning device.

20. The three-dimensional imaging system of claim 17, further comprising a three-dimensional printer, and the operations further comprising:
transforming the predicted three-dimensional volumetric model into a collection of machine control parameters for the three-dimensional printer;
providing the machine control parameters to the three-dimensional printer; and
depositing, by the three-dimensional printer, a tangible three-dimensional arrangement of physical material based on the predicted three-dimensional volumetric model.
